SEQUENCE LISTING (1 sequence)

SEQ ID NO: 1 - protein, 29 aa, Homo sapiens
HSQGTFTSDYSKYLDSRRAQDFVQWLMNT

(19) United States
(12) Patent Application Publication
(10) Pub. No.: US 2013/0053310 A1
(43) Pub. Date: Feb. 28, 2013

(54) NOVEL GLUCAGON ANALOGUES

(71) Applicants: Jesper F. Lau, Farum (DK); Thomas Kruse, Herlev (DK)

(72) Inventors: Jesper F. Lau, Farum (DK); Thomas Kruse, Herlev (DK)

(73) Assignee: NOVO NORDISK A/S, Bagsvaerd (DK)

(21) Appl. No.: 13/637,527

(22) PCT Filed: Mar. 28, 2011
(86) PCT No.: PCT/EP2011/054715

§ 371 Date: Nov. 8, 2012

Related U.S. Application Data
(60) Provisional application No. 61/319,962, filed on Apr. 1, 2010.

(30) Foreign Application Priority Data
Mar. 26, 2010 (EP) 10157900.1

(51) Int. Cl. C07K 14/605; A61P 3/08; A61K 38/26

(52) U.S. Cl. 514/6.8; 530/308; 514/11.7

(57) ABSTRACT

The present invention relates to glucagon peptide agonists which have improved solubility and stability, to the use of the peptides in therapy, to methods of treatment comprising administration of the peptides to patients in need thereof, and to the use of the peptides in the manufacture of medicaments. The glucagon peptides of the invention are of particular interest in relation to the treatment of hypoglycemia, diabetes and obesity, as well as a variety of diseases or conditions associated with hypoglycemia, diabetes and obesity.

### FIELD OF THE INVENTION

The present invention relates to novel glucagon peptide analogues with improved physical stability, to the use of said peptides in therapy, to methods of treatment comprising administration of said peptides to patients, and to the use of said peptides in the manufacture of medicaments.

### BACKGROUND OF THE INVENTION

The precise control of blood glucose levels is of vital importance to humans as well as other mammals. It is well established that the two hormones insulin and glucagon are important for maintenance of correct blood glucose levels. While insulin acts in the liver and peripheral tissues by reducing blood glucose levels via increased peripheral uptake of glucose and reduced glucose output from the liver, glucagon acts mainly on the pancreas and liver, by increasing blood glucose levels via up-regulation of gluconeogenesis and glycogenolysis. Glucagon has also been reported to increase lipolysis, to induce ketosis and to reduce plasma triglyceride levels in plasma [Schade and Eaton, Acta Diabetologica, 1977, 14, 62].

Human glucagon is a linear peptide 29 residues long and the distinctive combination of size and sequence of glucagon leads to considerable difficulties in handling the peptide in manufacture and in use. The molecule is too small to engage in productive and stabilizing tertiary structures, yet it is big enough to engage in phase transitions e.g to form beta-sheet like aggregates or fibrillar structures. Thus, in addition to the inherently low solubility of glucagon in the pH 3-9 range, the problem of handling glucagon in solution arises from rather small energy barriers separating the completely random conformation from the more distinctive structures including e.g. those necessary for binding to the receptor and those capable of forming fibrils. In handling of solutions of glucagon a choice must be made between acidic and basic formulations since the solubility is very low at pH 3-9. Due to the presence of several residues in native glucagon that are prone to base catalyzed deamidation, glucagon can only be handled for a short time at high pH (>10).

The commercial glucagon (Eli Lilly and Novo Nordisk) is supplied as a freeze-dried solid which must be dissolved prior to use. The solution must be injected shortly after preparation, since glucagon is prone to aggregation and fibrillation, when it is dissolved in simple aqueous formulations. Solutions of glucagon may become viscous or turbid a few hours after dissolution and consequently become unsuitable for injection. The dissolution process itself is a major problem for patients or relatives who may need to act quickly to counteract hypoglycemia since the patient may be mentally affected by hypoglycemia and relatives may be untrained in the dissolution process. In addition to the well-known use of glucagon for the treatment of acute hypoglycemia (e.g. “insulin shock”), the most important application is based on its spasmolytic effect on smooth muscles which is used clinically in connection with several imaging procedures, especially X-ray of the abdominal region.

Several patent applications disclosing different glucagon-based analogues and GLP-1/glucagon receptor co-agonists are known in the art, such as e.g. patents WO2008/086086, WO2008/101017, WO2007/056362, WO2008/152403 and WO96/29342. Some of the GLP-1/glucagon receptor co-agonists disclosed in these patents reffer to specific mutations relative to native human glucagon. Other glucagon analogs disclosed are PEGylated (e.g. WO2007/056362) or acylated in specific positions of native human glucagon (e.g. WO96/29342). Glucagon for prevention of hypoglycaemia has been disclosed, as e.g. in patent application U.S. Pat. No. 7,314,859.

The peptides of the present invention provide novel modified glucagon peptides with improved physical stability in solution.

### SUMMARY OF THE INVENTION

The present invention relates to novel glucagon peptides with improved physical stability in solution, to the use of said peptides in therapy to methods of treatment comprising administration of said peptides to patients, and to the use of said peptides in the manufacture of medicaments for use in the treatment of diabetes, obesity and related diseases and conditions, such as hypoglycemia.

In a first embodiment (Embodiment 1), the present invention relates to a glucagon peptide comprising SEQ ID 1, wherein the amino acid residue at position 25, i.e., X₂₅ represents His, Lys, Ile, Leu, Ala, Met, Cys, Asn, Val, Ser, Gln, Asp, Glu, Thr or (p)Tyr; or a pharmaceutically acceptable salt, amide, carboxylic acid or prodrug thereof.

The present invention further relates to the use of the compounds of the present invention in therapy, to pharmaceutical compositions comprising compounds of the invention and the use of the compounds of the invention in the manufacture of medicaments.

### DESCRIPTION OF THE DRAWINGS

FIG. 1 shows results from the ThT assay of glucagon analogue from Example 1, showing that the compound does not fibrillate upon physical stress.

FIG. 2 shows results from the ThT assay of glucagon analogue from Example 2. 1) 250 μM glucagon analogue from Example 2, 10 mM Hepes buffer pH 7.5; 2) 290 μM glucagon, 50 mM glycin pH 2.5 and 3) 1.45 mM glucagon, 50 mM glycin pH 2.5.

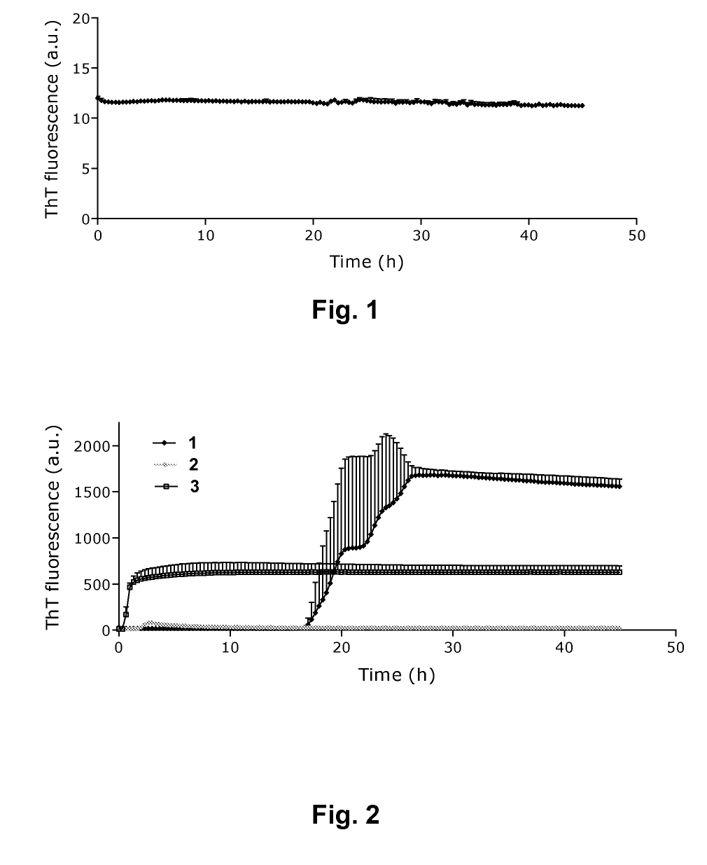

### DESCRIPTION OF THE INVENTION

The present invention relates to glucagon peptides with chemical modifications in position 25 that surprisingly improve the physical or chemical stability of said glucagon peptides in aqueous solution.

Among further embodiments of the present invention are the following:
2. The glucagon peptide according to embodiment 1, comprising the amino acid sequence of formula [I]:

[I]
His-Ser-Gln-Gly-Thr-Phe-Thr-Ser-Asp-Tyr-Ser-Lys-Tyr-Leu-Asp-Ser-Arg-Arg-Ala-
Gln-Asp-Phe-Val-Gln-X₂₅-Leu-Met-Asn-Thr.

3. The glucagon peptide according to any one of the previous embodiments, wherein X₂₅ represents His, Lys, or (p)Tyr.
4. The glucagon peptide according to any one of the previous embodiments, wherein X₂₅ represents Lys.
5. The glucagon peptide according to any one of the previous embodiments, wherein X₂₅ represents His.
6. The glucagon peptide according to any one of the previous embodiments, wherein X₂₅ represents (p)Tyr.
7. The glucagon peptide according to any one of the previous embodiments, wherein X₂₇ represents Leu.
8. The glucagon peptide according to any one of the previous embodiments, wherein X₂₅ represents Tyr and X₂₇ represents Leu.
9. The glucagon peptide according to embodiment 2, wherein X₂₅ represents Arg, with the proviso that X₂₇ represents Leu.
10. A glucagon peptide, wherein the amino acid residue at position 25, i.e., X₂₅ represents His, Arg, Lys, Ile, Leu, Ala, Met, Pro, Cys, Asn, Val, Ser, Gln, Asp, Glu, Thr or (p)Tyr;
   or a pharmaceutically acceptable salt, amide, carboxylic acid or prodrug thereof, wherein said wherein said glucagon peptide is an agonist of the glucagon receptor.
11. A glucagon peptide, wherein the amino acid residue at position 25, i.e., X₂₅ represents His, Arg, Lys, Ile, Leu, Ala, Met, Cys, Asn, Val, Ser, Gin, Asp, Glu, Thr or (p)Tyr; or a pharmaceutically acceptable salt, amide, carboxylic acid or prodrug thereof, wherein said wherein said glucagon peptide is an agonist of the glucagon receptor, with an EC₅₀<1 nM, such as 0.1 nM.
12. The glucagon peptide according to embodiments 10-11, comprising the amino acid sequence of formula [I]:

[I]
His-Ser-Gln-Gly-Thr-Phe-Thr-Ser-Asp-Tyr-Ser-Lys-Tyr-Leu-Asp-Ser-Arg-Arg-Ala-Gln-Asp-
Phe-Val-Gln-X₂₅-Leu-Met-Asn-Thr.

13. The glucagon peptide according to any one of embodiments 10-12, wherein X₂₅ represents His, Arg, Lys, or (p)Tyr.
14. The glucagon peptide according to any one of embodiments 10-12, wherein X₂₅ represents His.
15. The glucagon peptide according to any one of embodiments 10-12, wherein X₂₅ represents Arg.
16. A glucagon peptide according to any one of embodiments 10-12, wherein X₂₅ represents Lys.
17. The glucagon peptide according to any one of embodiments 10-12, wherein X₂₅ represents (p)Tyr.
18. The glucagon peptide according to any one of embodiments 10-12, wherein X₂₇ represents Leu.
19. The glucagon peptide according to any one of embodiments 10-12, wherein X₂₅ represents Tyr and X₂₇ represents Leu.
20. The glucagon peptide according to any one of embodiments 10-12, wherein X₂₅ represents Arg and X₂₇ represents Leu.
21. The glucagon peptide according to any one of the previous embodiments, wherein X₁₅ represents Glu.
22. The glucagon peptide according to any one of the previous embodiments, wherein X₂₁ represents Glu.
23. The glucagon peptide according to any one of the previous embodiments, wherein X₂₈ represents Ser or Glu.
24. The glucagon peptide according to any one of the previous embodiments, wherein X₂₈ represents Ser.
25. A glucagon peptide according to any one of the previous embodiments, wherein X₂₈ represents Glu.
26. A glucagon peptide, according to any one of the previous embodiments, selected from the group consisting of:

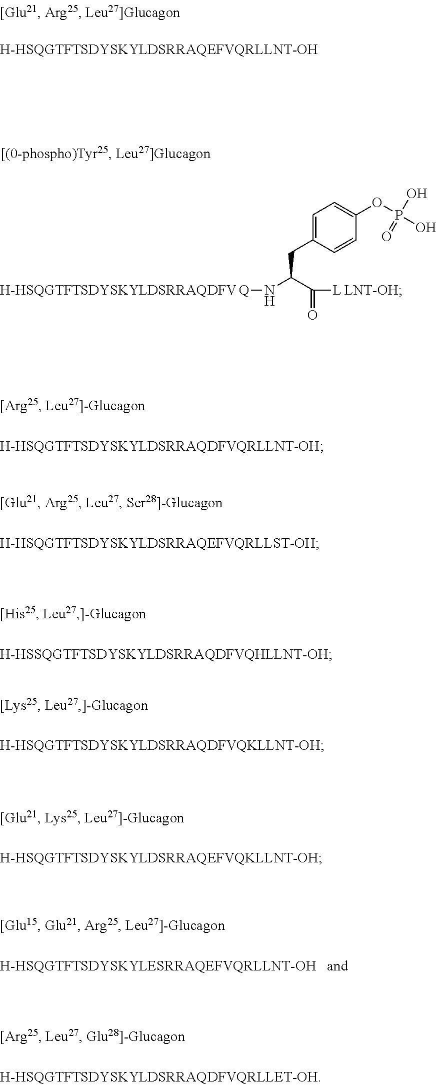

The present inventors surprisingly found glucagon-based compound which are improved compared to the present commercial products. These analogues could be used in a more safe and convenient prefilled ready-to-use device for the treatment of severe hypoglycaemia.

The present inventors surprisingly found that analogs of human glucagon with for example, mutation in amino acid position 25 of said glucagon peptide, are suitable for liquid formulation.

Mutations in the primary sequence of human glucagon(1-29) have been made using classical peptide synthesis. An assay as described in Assay (II) of the Pharmacological methods section, has been established whereby stability formulations of the new analogs were tested at pH 4, or at pH 7.5 by heating to 37° while shaking, and then measuring the fluorescence from an added dye (thioflavinT) that is known in the art to bind to fibrils and beta-sheet structures. When no ThT signal was observed within 45 hours and the analog was fully recovered by UPLC analysis, the analog was assumed to be fully stable.

When dissolved at acidic pH, glucagon can undergo various phase transitions that depend on concentration and temperature. After dissolving samples of glucagon in 0.01 N hydrochloric acid a lag-phase may occur where the viscosity of the sample is low and the solution is fully transparent. After some hours (Beaven et al, European J. Biochem. 11 (1969) 37-42) the viscosity begins to increase—indicative of a gelformation. After reaching a plateau, viscosity may begin to fall again and at the same time fibrils may appear and precipitate out of solution. The process is seedable, addition of a small amount of pre-formed gel reduce the lag-phase. Formation of gels and fibrillation is highly dependant of physical stress, such as heating and shaking, both increasing the rate of the process.

The inventors have also surprisingly found that the glucagon analogues of the present invention have improved stability towards formation of gels and fibrils in aqueous solutions. The stability of the compounds of the present invention may be measured by a method as described in Assay (II) of the Pharmacological Methods section.

The present invention relates to novel glucagon analogues with improved physical stability toward gel and fibril formation.

Further embodiments of the present invention relate to the following:

27. A glucagon peptide according to any one of the previous embodiments, wherein said glucagon peptide comprises up to five amino acid residues substitutions as compared to human glucagon (1-29).

28. A glucagon peptide according to any one of the previous embodiments, wherein said glucagon peptide comprises up to ten amino acid residues substitutions as compared to human glucagon (1-29).

29. A glucagon peptide according to any one of embodiments 27-28, wherein said substitutions are in the amino acid positions X₃, X₂₀, X₂₄, X₁₆, X₁₇, X₁₈, X₂₁, X₂₇, X₂₈ and/or X₂₉.

30. A glucagon peptide according to any one of embodiments 27-28, wherein said substitutions are in the amino acid positions X₁₇, X₁₈, X₂₁, X₂₇, X₂₈ and/or X₂₉.

31. A glucagon peptide according to any one of the previous embodiments, wherein said glucagon peptide comprises up to three amino acid residues substitutions when compared to human glucagon (1-29), wherein said substitutions are in the amino acid positions X₂₁, X₂₇, X₂₈ and/or X₂₉.

32. A glucagon peptide according to any one of the previous embodiments, wherein said glucagon peptide comprises up to three amino acid residues substitutions when compared to human glucagon (1-29), wherein said substitutions are in the amino acid positions X₂₇, X₂₈ and/or X₂₉.

33. A glucagon peptide according to any one of the previous embodiments, wherein said glucagon peptide is selected from glucagon (1-29), glucagon (1-29)-amide, or an analogue thereof.

34. A glucagon peptide according to any one of the previous embodiments, wherein said glucagon peptide comprises no more than fifteen amino acid residues which have been exchanged, added or deleted as compared to the sequence of human glucagon(1-29) or no more than ten amino acid residues which have been exchanged, added or deleted as compared to the sequence of human glucagon(1-29).

35. A glucagon peptide according to any one of the previous embodiments, wherein said glucagon peptide comprises no more than six amino acid residues which have been exchanged, added or deleted as compared to human glucagon(1-29).

36. A glucagon peptide according to any one of the previous embodiments, wherein said glucagon peptide comprises C-terminal extensions of up to three amino acid residues.

37. A glucagon peptide according to any one of the previous embodiments, wherein said glucagon peptide comprises C-terminal extensions of up to two amino acid residues.

38. A glucagon peptide according to any one of the previous embodiments, wherein said glucagon peptide comprises C-terminal extensions of one amino acid residue.

39. A glucagon peptide according to any one the previous embodiments, wherein the glucagon peptide is a C-terminal amide or a C-terminal carboxylic acid.

40. A glucagon peptide according to any one of the previous embodiments, wherein said glucagon peptide is a C-terminal amide.

41. A glucagon peptide according to any one of the previous embodiments, wherein said glucagon peptide is a C-terminal carboxylic acid.

In embodiments of the invention a maximum of 17 amino acids in the glucagon peptide have been modified (substituted, deleted, added or any combination thereof) relative to human glucagon(1-29). In embodiments of the invention a maximum of 15 amino acids in the glucagon peptide have been modified. In embodiments of the invention a maximum of 10 amino acids in the glucagon analogue have been modified. In embodiments of the invention a maximum of eight amino acids in the glucagon analogue have been modified. In embodiments of the invention a maximum of seven amino acids in the glucagon analogue have been modified. In embodiments of the invention a maximum of six amino acids in the glucagon analogue have been modified. In embodiments of the invention a maximum of five amino acids in the glucagon analogue have been modified. In embodiments of the invention a maximum of four amino acids in the glucagon analogue have been modified. In embodiments of the invention a maximum of three amino acids in the glucagon analogue have been modified. In embodiments of the invention a maximum of two amino acids in the glucagon analogue have been modified. In embodiments of the invention one amino acid in the glucagon analogue has been modified.

Further embodiments of the present invention relate to:

42. A glucagon peptide according to any of the previous embodiments, wherein said compound is an agonist of the glucagon receptor.

43. A glucagon peptide according to any of the previous embodiments, wherein said compound is an agonist of the glucagon receptor, with an EC₅₀<1 nM, such as 0.1nM.

44. A glucagon peptide according to any of the previous embodiments, wherein said glucagon peptide is a DPPIV protected compound.

45. A glucagon peptide according to any of the previous embodiments, wherein said glucagon peptide is DPPIV stabilised. 46. A glucagon peptide according to any of the previous embodiments, wherein said glucagon peptide has more than 70% recovery in the ThT fibrillation assay.

47. A glucagon peptide according to any of the previous embodiments, wherein said glucagon peptide has more than 90% recovery in the ThT fibrillation assay.

48. A glucagon peptide according to any of the previous embodiments, wherein said glucagon peptide has about 100% recovery in the ThT fibrillation assay.

49. A glucagon peptide according to any of the previous embodiments, wherein said glucagon peptide has more than 7 hours lag time in the ThT fibrillation assay.

50. A glucagon peptide according to any of the previous embodiments, wherein said glucagon peptide has more than 20 hours lag time in the ThT fibrillation assay.

51. A glucagon peptide according to any of the previous embodiments, wherein said glucagon peptide has 45 hours lag time or more in the ThT fibrillation assay.

The term “polypeptide” and “peptide” as used herein means a compound composed of at least five constituent amino acids connected by peptide bonds. The constituent amino acids may be from the group of the amino acids encoded by the genetic code and they may be natural amino acids which are not encoded by the genetic code, as well as synthetic amino acids. Natural amino acids which are not encoded by the genetic code are e.g. hydroxyproline, γ-carboxyglutamate, ornithine, phosphoserine, D-alanine and D-glutamine. Synthetic amino acids comprise amino acids manufactured by chemical synthesis, i.e. D-isomers of the amino acids encoded by the genetic code such as D-alanine and D-leucine, Aib (α-aminoisobutyric acid), Abu (α-aminobutyric acid), Tle (tert-butylglycine), β-alanine, 3-aminomethyl benzoic acid, anthranilic acid.

All amino acids for which the optical isomer is not stated is to be understood to mean the L-isomer.

The term “glucagon peptide” as used herein means glucagon peptide, glucagon compound, compound according to the present invention, compound of the present invention, compound of formula I, a glucagon analogue, a glucagon derivative or a derivative of a glucagon analogue human glucagon, human glucagon(1-29), glucagon(1-30), glucagon(1-31), glucagon(1-32) as well as analogues, fusion peptides, and derivatives thereof, which maintain glucagon activity.

As regards position numbering in glucagon compounds: for the present purposes any amino acid substitution, deletion, and/or addition is indicated relative to the sequences of native human glucagon (1-29) (SEQ ID 1). Human glucagon amino acids positions 1-29 are herein to be the same as amino acid positions X₁ to X₂₉. The human glucagon (1-29) sequence is His-Ser-Gln-Gly-Thr-Phe-Thr-Ser-Asp-Tyr-Ser-Lys-Tyr-Leu-AspvSer-Arg-Arg-Ala-Gln-Asp-Phe-Val-Gln-Trp-Leu-Met-Asn-Thr (SEQ ID 1).

Glucagon(1-30) means human glucagon with an extension of one amino acid in the C-terminal, glucagon(1-31) means human glucagon with an extension of two amino acid in the C-terminal and glucagon(1-32) means human glucagon with an extension of three amino acid in the C-terminal.

The term “DPP-IV protected” as used herein referring to a polypeptide means a polypeptide which has been chemically modified in order to render said compound resistant to the plasma peptidase dipeptidyl aminopeptidase-4 (DPP-IV). The DPP-IV enzyme in plasma is known to be involved in the degradation of several peptide hormones, e.g. glucagon, GLP-1, GLP-2, oxyntomodulin etc. Thus, a considerable effort is being made to develop analogues and derivatives of the polypeptides susceptible to DPP-IV mediated hydrolysis in order to reduce the rate of degradation by DPP-IV.

Furthermore, the glucagon peptides of the present invention are stabilized against DPP-IV cleavage in an albumin free assay as described in Assay III.

The term “glucagon agonist ” as used herein refers to any glucagon peptide which fully or partially activates the human glucagon receptor. In a preferred embodiment, the “glucagon agonist” is any glucagon peptide that binds to a glucagon receptor, preferably with an affinity constant (KD) or potency (EC₅₀) of below 1 μM, e.g., below 100 nM or 1 nM, as measured by methods known in the art such as Assay 1.

In the present context, the term “agonist” is intended to indicate a substance (ligand) that activates the receptor type in question.

In the present context, the term “antagonist” is intended to indicate a substance (ligand) that blocks, neutralizes or counteracts the effect of an agonist.

More specifically, receptor ligands may be classified as follows:

Receptor agonists, which activate the receptor; partial agonists also activate the receptor, but with lower efficacy than full agonists. A partial agonist will behave as a receptor partial antagonist, partially inhibiting the effect of a full agonist.

Receptor neutral antagonists, which block the action of an agonist, but do not affect the receptor-constitutive activity.

Receptor inverse agonists, which block the action of an agonist and at the same time attenuate the receptor-constitutive activity. A full inverse agonist will attenuate the receptor-constitutive activity completely; a partial inverse agonist will attenuate the receptor-constitutive activity to a lesser extent.

As used herein the term “antagonist” includes neutral antagonists and partial antagonists, as well as inverse agonists. The term “agonist” includes full agonists as well as partial agonists.

Other embodiments of the present relates to a pharmaceutical compositions:

52. A pharmaceutical composition comprising a glucagon peptide according to any one of the previous embodiments.

53. The pharmaceutical composition according to embodiment 52, further comprising one or more additional therapeutically active compounds or substances.

54. The pharmaceutical composition according to any one of embodiments 52-53, in unit dosage form comprising from about 0.001 mg to about 100 mg, such as from about 0.01 mg to about 10 mg, e.g. from about 0.1 mg to about 1 mg, of a glucagon peptide according to any of embodiments 1-51.

55. The pharmaceutical composition according to any one of embodiments 52-53, which is suited for parenteral administration.

56. A glucagon peptide according to any one of embodiments 1-51, for use in therapy.

In the present context, the term “pharmaceutically acceptable salt” is intended to indicate a salt which is not harmful to the patient. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric and nitric acids, and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene-salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. (1977) 66, 2, which is incorporated herein by reference. Examples of relevant metal salts include lithium, sodium, potassium and magnesium salts, and the like. Examples of alkylated ammonium salts include methylammonium, dimethylammonium, trimethylammonium, ethylammonium, hydroxyethylammonium, diethylammonium, butylammonium and tetramethylammonium salts, and the like.

As use herein, the term “therapeutically effective amount” of a compound refers to an amount sufficient to cure, alleviate or partially arrest the clinical manifestations of a given disease and/or its complications. An amount adequate to accomplish this is defined as a “therapeutically effective amount”. Effective amounts for each purpose will depend on the severity of the disease or injury, as well as on the weight and general state of the subject. It will be understood that determination of an appropriate dosage may be achieved using routine experimentation, by constructing a matrix of values and testing different points in the matrix, all of which is within the level of ordinary skill of a trained physician or veterinarian.

A typical dosage of a compound of the invention when employed in a method according to the present invention is in the range of from about 0.001 to about 100 mg/kg body weight per day, preferably from about 0.01 to about 10 mg/kg body weight, more preferably from about 0.01 to about 5 mg/kg body weight per day, e.g. from about 0.05 to about 10 mg/kg body weight per day or from about 0.03 to about 5 mg/kg body weight per day administered in one or more doses, such as from 1 to 3 doses. The exact dosage will depend upon the frequency and mode of administration, the sex, age, weight and general condition of the subject treated, the nature and severity of the condition treated, any concomitant diseases to be treated and other factors evident to those skilled in the art.

Compounds of the invention may conveniently be formulated in unit dosage form using techniques well known to those skilled in the art. A typical unit dosage form intended for oral administration one or more times per day, such as from one to three times per day, may suitably contain from about 0.05 to about 1000 mg, preferably from about 0.1 to about 500 mg, such as from about 0.5 to about 200 mg of a compound of the invention.

Compounds of the invention comprise compounds that are believed to be well-suited to administration with longer intervals than, for example, once daily, thus, appropriately formulated compounds of the invention may be suitable for, e.g., twice-weekly or once-weekly administration by a suitable route of administration, such as one of the routes disclosed herein.

Compounds of the present invention may be administered or applied in combination with one or more additional therapeutically active compounds or substances, and suitable additional compounds or substances may be selected, for example, from antidiabetic agents, antihyperlipidemic agents, antiobesity agents, antihypertensive agents and agents for the treatment of complications resulting from, or associated with, diabetes.

In the present context, if not stated otherwise, the terms “soluble”, “solubility”, “soluble in aquous solution”, “aqueous solubility”, “water soluble”, “water-soluble”, “water solubility”and “water-solubility”, refer to the solubility of a compound in water or in an aqueous salt or aqueous buffer solution, for example a 10 mM phosphate solution, or in an aqueous solution containing other compounds, but no organic solvents.

Further embodiments of the present invention relate to the following:

57. A glucagon peptide according to any of embodiments 1-5151, optionally in combination with one or more additional therapeutically active compounds, for use in treatment or prevention of hyperglycemia, type 2 diabetes, impaired glucose tolerance, type 1 diabetes and obesity

58. A glucagon peptide according to any of embodiments 1-51, optionally in combination with one or more additional therapeutically active compounds, for use in delaying or preventing disease progression in type 2 diabetes.

59. A glucagon peptide according to any of embodiments 1-51, optionally in combination with one or more additional therapeutically active compounds, for use treating obesity or preventing overweight.

60. A glucagon peptide according to any of embodiments 1-51, optionally in combination with one or more additional therapeutically active compounds, for use in for decreasing food intake.

61. A glucagon peptide according to any of embodiments 1-51, optionally in combination with one or more additional therapeutically active compounds, for use in increasing energy expenditure.

62. A glucagon peptide according to any of embodiments 1-51, optionally in combination with one or more additional therapeutically active compounds, for use in reducing body weight.

63. A glucagon peptide according to any of embodiments 1-51, optionally in combination with one or more additional therapeutically active compounds, for use in delaying the progression from impaired glucose tolerance (IGT) to type 2 diabetes.

64. A glucagon peptide according to any of embodiments 1-51, optionally in combination with one or more additional therapeutically active compounds, for use in delaying the progression from type 2 diabetes to insulin-requiring diabetes.

65. A glucagon peptide according to any of embodiments 1-51, optionally in combination with one or more additional therapeutically active compounds, for use in regulating appetite.

66. A glucagon peptide according to any of embodiments 1-51, optionally in combination with one or more additional therapeutically active compounds, for use inducing satiety.

67. A glucagon peptide according to any of embodiments 1-51, optionally in combination with one or more additional therapeutically active compounds, for use in preventing weight regain after successful weight loss.

68. A glucagon peptide according to any of embodiments 1-51, optionally in combination with one or more additional therapeutically active compounds, for use in treating a disease or state related to overweight or obesity.

69. A glucagon peptide according to any of embodiments 1-51, optionally in combination with one or more additional therapeutically active compounds, for use in treating bulimia.

70. A glucagon peptide according to any of embodiments 1-51, optionally in combination with one or more additional therapeutically active compounds, for use in treating binge-eating.

71. A glucagon peptide according to any of embodiments 1-51, optionally in combination with one or more additional therapeutically active compounds, for use in treating atherosclerosis.

72. A glucagon peptide according to any of embodiments 1-51, optionally in combination with one or more additional therapeutically active compounds, for use in treating hypertension.

73. A glucagon peptide according to any of embodiments 1-51, optionally in combination with one or more additional therapeutically active compounds, for use in treating type 2 diabetes.

74. A glucagon peptide according to any of embodiments 1-51, optionally in combination with one or more additional therapeutically active compounds, for use in treating impaired glucose tolerance.

75. A glucagon peptide according to any of embodiments 1-51, optionally in combination with one or more additional therapeutically active compounds, for use in treating dyslipidemia.

76. A glucagon peptide according to any of embodiments 1-51, optionally in combination with one or more additional therapeutically active compounds, for use in treating coronary heart disease.

77. A glucagon peptide according to any of embodiments 1-51, optionally in combination with one or more additional therapeutically active compounds, for use in treating hepatic steatosis.

78. A glucagon peptide according to any of embodiments 1-51, optionally in combination with one or more additional therapeutically active compounds, for use in treating hepatic steatosis.

79. A glucagon peptide according to any of embodiments 1-51, optionally in combination with one or more additional therapeutically active compounds, for use in treating beta-blocker poisoning.

80. A glucagon peptide according to any of embodiments 1-51, optionally in combination with one or more additional therapeutically active compounds, for use in inhibition of the motility of the gastrointestinal tract, useful in connection with investigations of the gastrointestinal tract using techniques such as x-ray, CT- and NMR-scanning.

81. A glucagon peptide according to any of embodiments 1-51, optionally in combination with one or more additional therapeutically active compounds, for use in treatment or prevention of hypoglycaemia.

82. A glucagon peptide according to any of embodiments 1-51, optionally in combination with one or more additional therapeutically active compounds, for use in treatment or prevention of insulin induced hypoglycaemia.

83. A glucagon peptide according to any of embodiments 1-51, optionally in combination with one or more additional therapeutically active compounds, for use in treatment or prevention of reactive hypoglycaemia.

84. A glucagon peptide according to any of embodiments 1-51, optionally in combination with one or more additional therapeutically active compounds, for use in treatment or prevention of diabetic hypoglycaemia.

85. A glucagon peptide according to any of embodiments 1-51, optionally in combination with one or more additional therapeutically active compounds, for use in treatment or prevention of non-diabetic hypoglycaemia.

86. A glucagon peptide according to any of embodiments 1-51, optionally in combination with one or more additional therapeutically active compounds, for use in treatment or prevention of fasting hypoglycaemia.

87. A glucagon peptide according to any of embodiments 1-51, optionally in combination with one or more additional therapeutically active compounds, for use in treatment or prevention of drug-induced hypoglycaemia.

88. A glucagon peptide according to any of embodiments 1-51, optionally in combination with one or more additional therapeutically active compounds, for use in treatment or prevention of gastric by-pass induced hypoglycaemia.

89. A glucagon peptide according to any of embodiments 1-51, optionally in combination with one or more additional therapeutically active compounds, for use in treatment or prevention of hypoglycemia in pregnancy.

90. A glucagon peptide according to any of embodiments 1-51, optionally in combination with one or more additional therapeutically active compounds, for use in treatment or prevention of alcohol-induced hypoglycaemia.

91. A glucagon peptide according to any of embodiments 1-51, optionally in combination with one or more additional therapeutically active compounds, for use in treatment or prevention of insulinoma.

92. A glucagon peptide according to any of embodiments 1-51, optionally in combination with one or more additional therapeutically active compounds, for use in treatment or prevention of Von Girkes disease.

Further embodiments of the present invention relate to the following methods:

93. A method for treating or preventing hyperglycemia, type 2 diabetes, impaired glucose tolerance, type 1 diabetes and obesity, comprising administering to a patient in need thereof, an effective amount of a compound according to any of embodiments 1-51, optionally in combination with one or more additional therapeutically active compounds.

94. A method for delaying or preventing disease progression in type 2 diabetes, comprising administering to a patient in need thereof, an effective amount of a compound according to any of embodiments 1-51, optionally in combination with one or more additional therapeutically active compounds.

95. A method for treating obesity or preventing overweight, comprising administering to a patient in need thereof, an effective amount of a compound according to any of embodiments 1-51, optionally in combination with one or more additional therapeutically active compounds.

96. A method for decreasing food intake, comprising administering to a patient in need thereof, an effective amount of a compound according to any of embodiments 1-51, optionally in combination with one or more additional therapeutically active compounds.

97. A method for use in increasing energy expenditure, comprising administering to a patient in need thereof, an effective amount of a compound according to any of embodiments 1-51, optionally in combination with one or more additional therapeutically active compounds.

98. A method for use in reducing body weight, comprising administering to a patient in need thereof, an effective amount of a compound according to any of embodiments 1-51, optionally in combination with one or more additional therapeutically active compounds.

99. A method for use in delaying the progression from impaired glucose tolerance (IGT) to type 2 diabetes, comprising administering to a patient in need thereof, an effective amount of a compound according to any of embodiments 1-51, optionally in combination with one or more additional therapeutically active compounds.

100. A method for use in delaying the progression from type 2 diabetes to insulin-requiring diabetes, comprising administering to a patient in need thereof, an effective amount of a compound according to any of embodiments 1-51, optionally in combination with one or more additional therapeutically active compounds.

101. A method for use in regulating appetite, comprising administering to a patient in need thereof, an effective amount of a compound according to any of embodiments 1-51, optionally in combination with one or more additional therapeutically active compounds.

102. A method for use in inducing satiety, comprising administering to a patient in need thereof, an effective amount of a compound according to any of embodiments 1-51, optionally in combination with one or more additional therapeutically active compounds.

103. A method for use in preventing weight regain after successful weight loss, comprising administering to a patient in need thereof, an effective amount of a compound according to any of embodiments 1-51, optionally in combination with one or more additional therapeutically active compounds.

104. A method for use in treating a disease or state related to overweight or obesity, comprising administering to a patient in need thereof, an effective amount of a compound according to any of embodiments 1-51, optionally in combination with one or more additional therapeutically active compounds.

105. A method for use in treating bulimia, comprising administering to a patient in need thereof, an effective amount of a compound according to any of embodiments 1-51, optionally in combination with one or more additional therapeutically active compounds.

106. A method for use in treating binge-eating, comprising administering to a patient in need thereof, an effective amount of a compound according to any of embodiments 1-51, optionally in combination with one or more additional therapeutically active compounds.

107. A method for use in treating atherosclerosis, comprising administering to a patient in need thereof, an effective amount of a compound according to any of embodiments 1-51, optionally in combination with one or more additional therapeutically active compounds.

108. A method for use in treating hypertension, comprising administering to a patient in need thereof, an effective amount of a compound according to any of embodiments 1-51, optionally in combination with one or more additional therapeutically active compounds.

109. A method for use in treating type 2 diabetes, comprising administering to a patient in need thereof, an effective amount of a compound according to any of embodiments 1-51, optionally in combination with one or more additional therapeutically active compounds.

110. A method for use in treating impaired glucose tolerance, comprising administering to a patient in need thereof, an effective amount of a compound according to any of embodiments 1-51, optionally in combination with one or more additional therapeutically active compounds.

111. A method for use in treating dyslipidemia, comprising administering to a patient in need thereof, an effective amount of a compound according to any of embodiments 1-51, optionally in combination with one or more additional therapeutically active compounds.

112. A method for use in treating coronary heart disease, comprising administering to a patient in need thereof, an effective amount of a compound according to any of embodiments 1-51, optionally in combination with one or more additional therapeutically active compounds.

113. A method for use in treating hepatic steatosis, comprising administering to a patient in need thereof, an effective amount of a compound according to any of embodiments 1-51, optionally in combination with one or more additional therapeutically active compounds.

114. A method for use in treating beta-blocker poisoning, comprising administering to a patient in need thereof, an effective amount of a compound according to any of embodiments 1-51, optionally in combination with one or more additional therapeutically active compounds.

115. A method for use in inhibition of the motility of the gastrointestinal tract, useful in connection with investigations of the gastrointestinal tract using techniques such as x-ray, CT- and NMR-scanning, comprising administering to a patient in need thereof, an effective amount of a compound according to any of embodiments 1-51, optionally in combination with one or more additional therapeutically active compounds.

116. A method for use in treatment or prevention of hypoglycaemia, comprising administering to a patient in need thereof, an effective amount of a compound according to any of embodiments 1-51, optionally in combination with one or more additional therapeutically active compounds.

117. A method for use in treatment or prevention of insulin induced hypoglycaemia, comprising administering to a patient in need thereof, an effective amount of a compound according to any of embodiments 1-51, optionally in combination with one or more additional therapeutically active compounds.

118. A method for use in treatment or prevention of reactive hypoglycaemia, comprising administering to a patient in need thereof, an effective amount of a compound according to any of embodiments 1-51, optionally in combination with one or more additional therapeutically active compounds.

119. A method for use in treatment or prevention of diabetic hypoglycaemia, comprising administering to a patient in need thereof, an effective amount of a compound according to any of embodiments 1-51, optionally in combination with one or more additional therapeutically active compounds. 120. A method for use in treatment or prevention of non-diabetic hypoglycaemia, comprising administering to a patient in need thereof, an effective amount of a compound according to any of embodiments 1-51, optionally in combination with one or more additional therapeutically active compounds.

121. A method for use in treatment or prevention of fasting hypoglycaemia, comprising administering to a patient in need thereof, an effective amount of a compound according to any of embodiments 1-51, optionally in combination with one or more additional therapeutically active compounds.

122. A method for use in treatment or prevention of drug-induced hypoglycaemia, comprising administering to a patient in need thereof, an effective amount of a compound according to any of embodiments 1-51, optionally in combination with one or more additional therapeutically active compounds.

123. A method for use in treatment or prevention of gastric by-pass induced hypoglycaemia, comprising administering to a patient in need thereof, an effective amount of a compound according to any of embodiments 1-51, optionally in combination with one or more additional therapeutically active compounds.

124. A method for use in treatment or prevention of hypoglycemia in pregnancy, comprising administering to a patient in need thereof, an effective amount of a compound according to any of embodiments 1-51, optionally in combination with one or more additional therapeutically active compounds.

125. A method for use in treatment or prevention of alcohol-induced hypoglycaemia, comprising administering to a patient in need thereof, an effective amount of a compound according to any of embodiments 1-51, optionally in combination with one or more additional therapeutically active compounds.

126. A method for use in treatment or prevention of insulinoma, comprising administering to a patient in need thereof, an effective amount of a compound according to any of embodiments 1-51, optionally in combination with one or more additional therapeutically active compounds.

127. A method for use in treatment or prevention of Von Girkes disease, comprising administering to a patient in need thereof, an effective amount of a compound according to any of embodiments 1-51, optionally in combination with one or more additional therapeutically active compounds.

Further embodiments of the present invention relate to the following uses:

128. Use of a glucagon peptide according to any one of the embodiments 1-51, for the preparation of a medicament.

129. Use of a glucagon peptide according to any one of embodiments 1-51, for the preparation of a medicament for the treatment or prevention of hyperglycemia, type 2 diabetes, impaired glucose tolerance, type 1 diabetes and obesity.

130. Use of a glucagon peptide according to any one of the embodiments 1-51, for the preparation of a medicament for delaying or preventing disease progression in type 2 diabetes, treating obesity or preventing overweight, for decreasing food intake, increase energy expenditure, reducing body weight, delaying the progression from impaired glucose tolerance (IGT) to type 2 diabetes; delaying the progression from type 2 diabetes to insulin-requiring diabetes; regulating appetite; inducing satiety; preventing weight regain after successful weight loss; increasing energy expenditure; treating a disease or state related to overweight or obesity; treating bulimia; treating binge-eating; treating atherosclerosis, hypertension, type 2 diabetes, IGT, dyslipidemia, coronary heart disease, hepatic steatosis, treatment of beta-blocker poisoning, use for inhibition of the motility of the gastrointestinal tract, useful in connection with investigations of the gastrointestinal tract using techniques such as x-ray, CT- and NMR-scanning.

131. Use of a glucagon peptide according to any one of the embodiments 1-51, for the preparation of a medicament for treatment or prevention of hypoglycemia, insulin induced hypoglycemia, reactive hypoglycemia, diabetic hypoglycemia, non-diabetic hypoglycemia, fasting hypoglycemia, drug-induced hypoglycemia, gastric by-pass induced hypoglycemia, hypoglycemia in pregnancy, alcohol induced hypoglycemia, insulinoma and Von Girkes disease.

Other embodiments of the present invention relate to the following:

132. A glucagon peptide(1-29) agonist, wherein the amino acid residue at position 25, i.e., wherein

X₂₅ represents His, Arg, Lys, Ile, Phe, Leu, Ala, Met, Pro, Cys, Asn, Val, Gly, Ser, Gln, Tyr, Asp, Glu, Thr or (p)Tyr;
or a pharmaceutically acceptable salt, amide, carboxylic acid or prodrug thereof.

133. A glucagon peptide according to embodiment 132, comprising the amino acid sequence of formula [I]:

[I]
His-Ser-Gln-Gly-Thr-Phe-Thr-Ser-Asp-Tyr-Ser-Lys-Tyr-Leu-Asp-Ser-Arg-Arg-Ala-Gln-Asp-
Phe-Val-Gln-X₂₅-Leu-Met-Asn-Thr.

134. A compound according to embodiments 132-133, selected from the group consisting of:

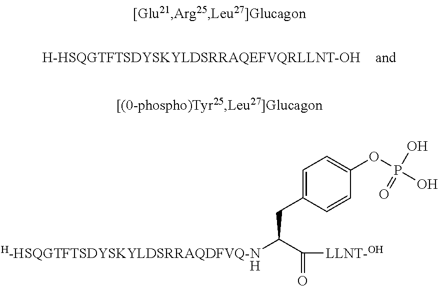

135. A glucagon peptide according to any one of embodiments 132-134, wherein X₂₅ represents His, Arg, Lys, Glu, Asp, Gly, Pro, Tyr or (p)Tyr.

136. A glucagon peptide according to embodiment 132, wherein X₂₅ represents Arg or Tyr.

137. A glucagon peptide according to embodiment 132, wherein X₂₅ represents Arg.

138. A glucagon peptide according to embodiment 132, wherein X₂₅ represents Tyr.

139. A pharmaceutical composition comprising a peptide according to any one of embodiments 132-133.

140. A pharmaceutical composition according to embodiment 139, further comprising one or more additional therapeutically active compounds or substances.

141. The pharmaceutical composition according to any one of embodiments 139-140, which is suited for parenteral administration.

142. A peptide according to any of any one of embodiments 132-133, for use in therapy.

143. Use of a peptide according to any one of the embodiments 132-133, for the preparation of a medicament.

144. Use of a peptide according to any one of embodiments 132-133, for the preparation of a medicament for the treatment or prevention of hyperglycemia, type 2 diabetes, impaired glucose tolerance, type 1 diabetes and obesity.

145. Use of a peptide according to any one of the embodiments 132-133, for the preparation of a medicament for delaying or preventing disease progression in type 2 diabetes, treating obesity or preventing overweight, for decreasing food intake, increase energy expenditure, reducing body weight, delaying the progression from impaired glucose tolerance (IGT) to type 2 diabetes; delaying the progression from type 2 diabetes to insulin-requiring diabetes; regulating appetite; inducing satiety; preventing weight regain after successful weight loss; treating a disease or state related to overweight or obesity; treating bulimia; treating binge-eating; treating atherosclerosis, hypertension, type 2 diabetes, IGT, use for inhibition of the motility of the gastrointestinal tract, useful in connection with investigations of the gastrointestinal tract using techniques such as x-ray, CT- and NMR-scanning.

146. Use of a peptide according to any one of embodiments 132-133, for the preparation of a medicament for reatment or prevention of hypoglycemia, insulin induced hypoglycemia, reactive hypoglycemia, diabetic hypoglycemia, non-diabetic hypoglycemia, fasting hypoglycemia, drug-induced hypoglycemia, gastric by-pass induced hypoglycemia, hypoglycemia in pregnancy, alcohol induced hypoglycemia, insulinoma and Von Girkes disease.

The terms “treatment”, “treating” and other variants thereof as used herein refer to the management and care of a patient for the purpose of combating a condition, such as a disease or a disorder. The terms are intended to include the full spectrum of treatments for a given condition from which the patient is suffering, such as administration of the active compound(s) in question to alleviate symptoms or complications thereof, to delay the progression of the disease, disorder or condition, to cure or eliminate the disease, disorder or condition, and/or to prevent the condition, in that prevention is to be understood as the management and care of a patient for the purpose of combating the disease, condition, or disorder, and includes the administration of the active compound(s) in question to prevent the onset of symptoms or complications. The patient to be treated is preferably a mammal, in particular a human being, but treatment of other animals, such as dogs, cats, cows, horses, sheep, goats or pigs, is within the scope of the invention.

The term “obesity” implies an excess of adipose tissue. When energy intake exceeds energy expenditure, the excess calories are stored in adipose tissue, and if this net positive balance is prolonged, obesity results, i.e. there are two components to weight balance, and an abnormality on either side (intake or expenditure) can lead to obesity. In this context, obesity is best viewed as any degree of excess adipose tissue that imparts a health risk. The distinction between normal and obese individuals can only be approximated, but the health risk imparted by obesity is probably a continuum with increasing adipose tissue. However, in the context of the present invention, individuals with a body mass index (BMI=body weight in kilograms divided by the square of the height in meters) above 25 are to be regarded as obese.

In certain embodiments of the uses and methods of the present invention, the compound of the present invention may be administered or applied in combination with more than one of the above-mentioned, suitable additional therapeutically active compounds or substances, e.g. in combination with: metformin and a sulfonylurea such as glyburide; a sulfonylurea and acarbose; nateglinide and metformin; acarbose and metformin; a sulfonylurea, metformin and troglitazone; insulin and a sulfonylurea; insulin and metformin; insulin, metformin and a sulfonylurea; insulin and troglitazone; insulin and lovastatin; etc.

The amino acid abbreviations used in the present context have the following meanings:

| | |
|---|---|
| Aib | 2-Aminoisobutyric acid |
| Ala | Alanine |
| Asn | Asparagine |
| Asp | Aspartic acid |
| Arg | Arginine |
| Cit | Citrulline |
| Cys | Cysteine |
| Gln | Glutamine |
| Glu | Glutamic acid |
| Gly | Glycine |
| His | Histidine |
| Hyp | 4-hydroxyproline |
| Ile | Isoleucine |
| Leu | Leucine |
| Lys | Lysine |
| Met | Methionine |
| Orn | Ornithine |
| Phe | Phenylalanine |
| Pro | Proline |
| Ser | Serine |
| Thr | Threonine |
| Tyr | Tyrosine |
| p(Tyr) | 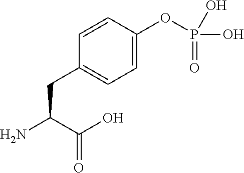 [SMILES: N[C@@H](Cc1ccc(OP(=O)(O)O)cc1)C(=O)O] |
| Trp | Tryptophan |
| Val | Valine |

Amino acid abbreviations beginning with D- followed by a three letter code, such as D-Ser, D-His and so on, refer to the D-enantiomer of the corresponding amino acid, for example D-serine, D-histidine and so on.

### Pharmaceutical Compositions

Pharmaceutical compositions containing a compound according to the present invention may be prepared by conventional techniques, e.g. as described in Remington's Pharmaceutical Sciences, 1985 or in Remington: The Science and Practice of Pharmacy, 19^{th} edition, 1995.

As already mentioned, one aspect of the present invention is to provide a pharmaceutical formulation comprising a compound according to the present invention which is present in a concentration from about 0.01 mg/ml to about 25 mg/ml, such as from about 1 mg/mL to about 10 mg/mL, from about 1 mg/mL to about 5 mg/mL and wherein said formulation has a pH from 2.0 to 10.0. The pharmaceutical formulation may comprise a compound according to the present invention which is present in a concentration from about 0.01 mg/ml to about 50 mg/ml, and wherein said formulation has a pH from 2.0 to 10.0. The formulation may further comprise a buffer system, preservative(s), isotonicity agent(s), chelating agent(s), stabilizers and surfactants. In one embodiment of the invention the pharmaceutical formulation is an aqueous formulation, i.e. formulation comprising water. Such formulation is typically a solution or a suspension. In a further embodiment of the invention the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In a further aspect the invention relates to a pharmaceutical formulation comprising an aqueous solution of a compound according to the present invention, and a buffer, wherein said compound is present in a concentration from 0.1 mg/ml or above, and wherein said formulation has a pH from about 2.0 to about 10.0.

In a further aspect the invention relates to a pharmaceutical formulation comprising an aqueous solution of a compound according to the present invention, and a buffer, wherein said compound is present in a concentration from 0.1 mg/ml or above, and wherein said formulation has a pH from about 7.0 to about 8.5.

In a another embodiment of the invention the pH of the formulation is selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and 10.0. Preferably, the pH of the formulation is at least 1 pH unit from the isoelectric point of the compound according to the present invention, even more preferable the pH of the formulation is at least 2 pH units from the isoelectric point of the compound according to the present invention.

In a further embodiment of the invention the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethane, hepes, bicine, tricine, malic acid, succinate, maleic acid, fumaric acid, tartaric acid, aspartic acid or mixtures thereof. Each one of these specific buffers constitutes an alternative embodiment of the invention.

In a further embodiment of the invention the formulation further comprises a pharmaceutically acceptable preservative. In a further embodiment of the invention the preservative is selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, ethanol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3(p-chlorphenoxy)propane-1,2-diol) or mixtures thereof. In a further embodiment of the invention the preservative is present in a concentration from 0.1 mg/ml to 30 mg/ml. In a further embodiment of the invention the preservative is present in a concentration from 0.1 mg/ml to 20 mg/ml. In a further embodiment of the invention the preservative is present in a concentration from 0.1 mg/ml to 5 mg/ml. In a further embodiment of the invention the preservative is present in a concentration from 5 mg/ml to 10 mg/ml. In a further embodiment of the invention the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 19^{th} edition, 1995.

In a further embodiment of the invention the formulation further comprises an isotonic agent. In a further embodiment of the invention the isotonic agent is selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol is defined as a C4-C8 hydrocarbon having at least one —OH group and includes, for example, mannitol, sorbitol, inositol, galacititol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects achieved using the methods of the invention. In one embodiment, the sugar or sugar alcohol concentration is between about 1 mg/ml and about 150 mg/ml. In a further embodiment of the invention the isotonic agent is present in a concentration from 1 mg/ml to 50 mg/ml. In a further embodiment of the invention the isotonic agent is present in a concentration from 1 mg/ml to 7 mg/ml. In a further embodiment of the invention the isotonic agent is present in a concentration from 8 mg/ml to 24 mg/ml. In a further embodiment of the invention the isotonic agent is present in a concentration from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 19^{th} edition, 1995.

In a further embodiment of the invention the formulation further comprises a chelating agent. In a further embodiment of the invention the chelating agent is selected from salts of ethylenediaminetetraacetic acid (EDTA), citric acid, and aspartic acid, and mixtures thereof. In a further embodiment of the invention the chelating agent is present in a concentration from 0.1 mg/ml to 5 mg/ml. In a further embodiment of the invention the chelating agent is present in a concentration from 0.1 mg/ml to 2 mg/ml. In a further embodiment of the invention the chelating agent is present in a concentration from 2 mg/ml to 5 mg/ml. Each one of these specific chelating agents constitutes an alternative embodiment of the invention. The use of a chelating agent in pharmaceutical compositions is well-known to the skilled person. For convenience reference is made to Remington: The Science and Practice of Pharmacy, 19^{th} edition, 1995.

In a further embodiment of the invention the formulation further comprises a stabiliser. The use of a stabilizer in pharmaceutical compositions is well-known to the skilled person. For convenience reference is made to Remington: The Science and Practice of Pharmacy, 19^{th} edition, 1995.

More particularly, compositions of the invention are stabilized liquid pharmaceutical compositions whose therapeutically active components include a polypeptide that possibly exhibits aggregate formation during storage in liquid pharmaceutical formulations. By “aggregate formation” is intended a physical interaction between the polypeptide molecules that results in formation of oligomers, which may remain soluble, or large visible aggregates that precipitate from the solution. By “during storage” is intended a liquid pharmaceutical composition or formulation once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or formulation is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53). Aggregate formation by a polypeptide during storage of a liquid pharmaceutical composition can adversely affect biological activity of that polypeptide, resulting in loss of therapeutic efficacy of the pharmaceutical composition. Furthermore, aggregate formation may cause other problems such as blockage of tubing, membranes, or pumps when the polypeptide-containing pharmaceutical composition is administered using an infusion system.

The pharmaceutical compositions of the invention may further comprise an amount of an amino acid base sufficient to decrease aggregate formation by the polypeptide during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids used for preparing the compositions of the invention are those carrying a charged side chain, such as arginine, lysine, aspartic acid, and glutamic acid. In one embodiment, the amino acid used for preparing the compositions of the invention is glycine. Any stereoisomer (i.e. L or D) of a particular amino acid (e.g. methionine, histidine, imidazole, arginine, lysine, isoleucine, aspartic acid, tryptophan, threonine and mixtures thereof) or combinations of these stereoisomers, may be present in the pharmaceutical compositions of the invention so long as the particular amino acid is present either in its free base form or its salt form. In one embodiment the L-stereoisomer is used. Compositions of the invention may also be formulated with analogues of these amino acids. By “amino acid analogue” is intended a derivative of the naturally occurring amino acid that brings about the desired effect of decreasing aggregate formation by the polypeptide during storage of the liquid pharmaceutical compositions of the invention. Suitable arginine analogues include, for example, aminoguanidine, ornithine and N-monoethyl L-arginine, suitable methionine analogues include ethionine and buthionine and suitable cystein analogues include S-methyl-L cystein. As with the other amino acids, the amino acid analogues are incorporated into the compositions in either their free base form or their salt form. In a further embodiment of the invention the amino acids or amino acid analogues are used in a concentration, which is sufficient to prevent or delay aggregation of the protein.

In a further embodiment of the invention methionine (or other sulphuric amino acids or amino acid analogous) may be added to inhibit oxidation of methionine residues to methionine sulfoxide when the polypeptide acting as the therapeutic agent is a polypeptide comprising at least one methionine residue susceptible to such oxidation. By “inhibit” is intended minimal accumulation of methionine oxidized species over time. Inhibiting methionine oxidation results in greater retention of the polypeptide in its proper molecular form. Any stereoisomer of methionine (L, D or a mixture thereof) can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be achieved by adding methionine such that the ratio of methionine added to methionine residues ranges from about 1:1 to about 1000:1, such as 10:1 to about 100:1.

In a further embodiment of the invention the formulation further comprises a stabiliser selected from the group of high molecular weight polymers or low molecular compounds. In a further embodiment of the invention the stabilizer is selected from polyethylene glycol (e.g. PEG 3350), polyvinylalcohol (PVA), polyvinylpyrrolidone, carboxy-/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative embodiment of the invention.

The pharmaceutical compositions may also comprise additional stabilizing agents, which further enhance stability of a therapeutically active polypeptide therein. Stabilizing agents of particular interest to the present invention include, but are not limited to, methionine and EDTA, which protect the polypeptide against methionine oxidation, and a nonionic surfactant, which protects the polypeptide against aggregation associated with freeze-thawing or mechanical shearing.

In a further embodiment of the invention the formulation further comprises a surfactant. In a further embodiment of the invention the surfactant is selected from a detergent, ethoxylated castor oil, polyglycolyzed glycerides, acetylated monoglycerides, sorbitan fatty acid esters, polyoxypropylene-polyoxyethylene block polymers (eg. poloxamers such as Pluronic® F68, poloxamer 188 and 407, Triton X-100), polyoxyethylene

sorbitan fatty acid esters, starshaped PEO, polyoxyethylene and polyethylene derivatives such as alkylated and alkoxylated derivatives (tweens, e.g. Tween-20, Tween-40, Tween-80 and Brij-35), polyoxyethylene hydroxystearate, monoglycerides or ethoxylated derivatives thereof, diglycerides or polyoxyethylene derivatives thereof, alcohols, glycerol, lecitins and phospholipids (eg. phosphatidyl serine, phosphatidyl choline, phosphatidyl ethanolamine, phosphatidyl inositol, diphosphatidyl glycerol and sphingomyelin), derivates of phospholipids (eg. dipalmitoyl phosphatidic acid) and lysophospholipids (eg. palmitoyl lysophosphatidyl-L-serine and 1-acyl-sn-glycero-3-phosphate esters of ethanolamine, choline, serine or threonine) and alkyl, alkoxyl (alkyl ester), alkoxy (alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (eg. galactopyransoide), sphingoglycolipids (eg. ceramides, gangliosides), dodecylphosphocholine, hen egg lysolecithin, fusidic acid derivatives-(e.g. sodium tauro-dihydrofusidate etc.), long-chain fatty acids and salts thereof C6-C12 (eg. oleic acid and caprylic acid), acylcarnitines and derivatives, N^{α}-acylated derivatives of lysine, arginine or histidine, or side-chain acylated derivatives of lysine or arginine, N^{α}-acylated derivatives of dipeptides comprising any combination of lysine, arginine or histidine and a neutral or acidic amino acid, N^{α}-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulfate or sodium lauryl sulfate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quarternary ammonium bases) (e.g. cetyl-trimethylammonium bromide, cetylpyridinium chloride), non-ionic surfactants (eg. Dodecyl β-D-glucopyranoside), poloxamines (eg. Tetronic's), which are tetrafunctional block copolymers derived from sequential addition of propylene oxide and ethylene oxide to ethylenediamine, or the surfactant may be selected from the group of imidazoline derivatives, or mixtures thereof. Each one of these specific surfactants constitutes an alternative embodiment of the invention.

The use of a surfactant in pharmaceutical compositions is well-known to the skilled person. For convenience reference is made to Remington: The Science and Practice of Pharmacy, 19^{th} edition, 1995.

Additional ingredients may also be present in the pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatin or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Pharmaceutical compositions containing a compound according to the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the compound, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behaviour in lipid-water systems, polymeric micelles, multiple emulsions, self-emulsifying, self-microemulsifying, cyclodextrins and derivatives thereof, and dendrimers.

Compositions of the current invention are useful in the formulation of solids, semisolids, powder and solutions for pulmonary administration of the compound, using, for example a metered dose inhaler, dry powder inhaler and a nebulizer, all being devices well known to those skilled in the art.

Compositions of the current invention are specifically useful in the formulation of controlled, sustained, protracting, retarded, and slow release drug delivery systems. More specifically, but not limited to, compositions are useful in formulation of parenteral controlled release and sustained release systems (both systems leading to a many-fold reduction in number of administrations), well known to those skilled in the art. Even more preferably, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles,

Methods to produce controlled release systems useful for compositions of the current invention include, but are not limited to, crystallization, condensation, co-cystallization, precipitation, co-precipitation, emulsification, dispersion, high pressure homogenization, encapsulation, spray drying, microencapsulation, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Formulation and Delivery (MacNally, E. J., ed. Marcel Dekker, New York, 2000).

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the compound according to the present invention in the form of a nasal or pulmonal spray. As a still further option, the pharmaceutical compositions containing the compound of the invention can also be adapted to transdermal administration, e.g. by needle-free injection or from a patch, optionally an iontophoretic patch, or transmucosal, e.g. buccal, administration.

The term “stabilized formulation” refers to a formulation with increased physical stability, increased chemical stability or increased physical and chemical stability.

The term “physical stability” of the protein formulation as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the aqueous protein formulations is evaluated by means of visual inspection and/or turbidity measurements after exposing the formulation filled in suitable containers (e.g. cartridges or vials) to mechanical/physical stress (e.g. agitation) at different temperatures for various time periods. Visual inspection of the formulations is performed in a sharp focused light with a dark background. The turbidity of the formulation is characterized by a visual score ranking the degree of turbidity for instance on a scale from 0 to 3 (a formulation showing no turbidity corresponds to a visual score 0, and a formulation showing visual turbidity in daylight corresponds to visual score 3). A formulation is classified physical unstable with respect to protein aggregation, when it shows visual turbidity in daylight. Alternatively, the turbidity of the formulation can be evaluated by simple turbidity measurements well-known to the skilled person. Physical stability of the aqueous protein formulations can also be evaluated by using a spectroscopic agent or probe of the conformational status of the protein. The probe is preferably a small molecule that preferentially binds to a non-native conformer of the protein. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as antrhacene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the protein formulation as used herein refers to chemical covalent changes in the protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the protein formulation as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation (of for instance methionine residues) can be mentioned as another variant of chemical degradation. The chemical stability of the protein formulation can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

Hence, as outlined above, a “stabilized formulation” refers to a formulation with increased physical stability, increased chemical stability or increased physical and chemical stability. In general, a formulation must be stable during use and storage (in compliance with recommended use and storage conditions) until the expiration date is reached.

In one embodiment of the invention the pharmaceutical formulation comprising the compound according to the present invention is stable for more than 6 weeks of usage and for more than 3 years of storage.

In another embodiment of the invention the pharmaceutical formulation comprising the compound according to the present invention is stable for more than 4 weeks of usage and for more than 3 years of storage.

In a further embodiment of the invention the pharmaceutical formulation comprising the compound according to the present invention is stable for more than 4 weeks of usage and for more than two years of storage.

In a further embodiment of the invention the pharmaceutical formulation comprising the compound is stable for more than 2 weeks of usage and for more than two years of storage.

In a further embodiment of the invention the pharmaceutical formulation comprising the compound is stable for more than 2 weeks of usage and for more than one year of storage.

In a further embodiment of the invention the pharmaceutical formulation comprising the compound is stable for more than 2 weeks of usage and for more than 6 months of storage.

Pharmaceutical compositions containing a glucagon peptide according to the present invention may be administered parenterally to patients in need of such a treatment. Parenteral administration may be performed by subcutaneous, intramuscular or intravenous injection by means of a syringe, optionally a pen-like syringe. Alternatively, parenteral administration can be performed by means of an infusion pump. A further option is a composition which may be a powder or a liquid for the administration of the glucagon peptide in the form of a nasal or pulmonal spray. As a still further option, the glucagon peptides of the invention can also be administered transdermally, e.g. from a patch, optionally a iontophoretic patch, or transmucosally, e.g. bucally.

Thus, the injectable compositions of the glucagon peptide of the present invention can be prepared using the conventional techniques of the pharmaceutical industry which involves dissolving and mixing the ingredients as appropriate to give the desired end product.

According to one embodiment of the present invention, the glucagon peptide is provided in the form of a composition suitable for administration by injection. Such a composition can either be an injectable solution ready for use or it can be an amount of a solid composition, e.g. a lyophilised product, which has to be dissolved in a solvent before it can be injected.

The glucagon peptides of this invention can be used in the treatment of various diseases. The particular glucagon peptide to be used and the optimal dose level for any patient will depend on the disease to be treated and on a variety of factors including the efficacy of the specific peptide derivative employed, the age, body weight, physical activity, and diet of the patient, on a possible combination with other drugs, and on the severity of the case. It is recommended that the dosage of the glucagon peptide of this invention be determined for each individual patient by those skilled in the art.

The treatment with a compound according to the present invention may also be combined with combined with a second or more pharmacologically active substances, e.g. selected from antidiabetic agents, antiobesity agents, appetite regulating agents, antihypertensive agents, agents for the treatment and/or prevention of complications resulting from or associated with diabetes and agents for the treatment and/or prevention of complications and disorders resulting from or associated with obesity. In the present context the expression “antidiabetic agent” includes compounds for the treatment and/or prophylaxis of insulin resistance and diseases wherein insulin resistance is the pathophysiological mechanism.

Examples of these pharmacologically active substances are: Insulin, GLP-1 agonists, sulphonylureas (e.g. tolbutamide, glibenclamide, glipizide and gliclazide), biguanides e.g. metformin, meglitinides, glucosidase inhibitors (e.g. acorbose), glucagon antagonists, DPP-IV (dipeptidyl peptidase-IV) inhibitors, inhibitors of hepatic enzymes involved in stimulation of gluconeogenesis and/or glycogenolysis, glucose uptake modulators, thiazolidinediones such as troglitazone and ciglitazone, compounds modifying the lipid metabolism such as antihyperlipidemic agents as HMG CoA inhibitors (statins), compounds lowering food intake, RXR agonists and agents acting on the ATP-dependent potassium channel of the β-cells, e.g. glibenclamide, glipizide, gliclazide and repaglinide; Cholestyramine, colestipol, clofibrate, gemfibrozil, lovastatin, pravastatin, simvastatin, probucol, dextrothyroxine, neteglinide, repaglinide; β-blockers such as alprenolol, atenolol, timolol, pindolol, propranolol and metoprolol, ACE (angiotensin converting enzyme) inhibitors such as benazepril, captopril, enalapril, fosinopril, lisinopril, alatriopril, quinapril and ramipril, calcium channel blockers such as nifedipine, felodipine, nicardipine, isradipine, nimodipine, diltiazem and verapamil, and a-blockers such as doxazosin, urapidil, prazosin and terazosin; CART (cocaine amphetamine regulated transcript) agonists, NPY (neuropeptide Y) antagonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, β3 agonists, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, RXR (retinoid X receptor) modulators, TR β agonists; histamine H3 antagonists.

It should be understood that any suitable combination of the compounds according to the invention with one or more of the above-mentioned compounds and optionally one or more further pharmacologically active substances are considered to be within the scope of the present invention.

The present invention is further illustrated by the following examples which, however, are not to be construed as limiting the scope of protection. The features disclosed in the foregoing description and in the following examples may, both separately and in any combination thereof, be material for realizing the invention in diverse forms thereof.

### EXAMPLES

### List of Abbreviations

DCM: Dichloromethane
DIC: Diisopropylcarbodiimide
Fmoc: 9-fluorenylmethyloxycarbonyl
HOAt: 1-hydroxy-7-azabenzotriazole
HPLC: High Performance Liquid Chromatography
LCMS: Liquid Chromatography Mass Spectroscopy
MeCN Acetonitrile
NMP: N-methyl pyrrolidone
RP: Reverse Phase
RP-HPLC: Reverse Phase High Performance Liquid Chromatography
RT: Room Temperature
Rt: Retention time
SPPS: Solid Phase Peptide Synthesis
TFA: Trifluoroacetic acid
TIPS: Triisopropylsilane
UPLC: Ultra Performance Liquid Chromatography

### General Methods

This section relates to methods for synthesising resin bound peptide (SPPS methods, including methods for de-protection of amino acids, methods for cleaving the peptide from the resin, and for its purification), as well as methods for detecting and characterising the resulting peptide (LCMS and UPLC methods).

### SPPS General Methods

The Fmoc-protected amino acid derivatives used were the standard recommended: Fmoc-Ala-OH, Fmoc-Arg(Pbf)-OH, Fmoc-Asn(Trt)-OH, Fmoc-Asp(OtBu)-OH, Fmoc-Cys(Trt)-OH, Fmoc-Gln(Trt)-OH, Fmoc-Glu(OtBu)-OH, Fmoc-Gly-OH, Fmoc-His(Trt)-OH, Fmoc-Ile-OH, Fmoc-Leu-OH, Fmoc-Lys(BOC)-OH, Fmoc-Met-OH, Fmoc-Phe-OH, Fmoc-Pro-OH, Fmoc-Ser(tBu)-OH, Fmoc-Thr(tBu)-OH, Fmoc-Trp(BOC)-OH, Fmoc-Tyr(tBu)-OH and Fmoc-Val-OH, supplied from e.g. Anaspec, Bachem, Iris Biotech, or NovabioChem. SPPS were performed using Fmoc based chemistry on a Prelude Solid Phase Peptide Synthesizer from Protein Technologies (Tucson, Ariz. 85714 U.S.A.). A suitable resin for the preparation of C-terminal carboxylic acids is a pre-loaded, low-load Wang resin available from NovabioChem (e.g. low load fmoc-Thr(tBu)-Wang resin, LL, 0.27 mmol/g). A suitable resin for the synthesis of glucagon analogues with a C-terminal amide is PAL-ChemMatrix resin available from Matrix-Innovation. The N-terminal alpha amino group was protected with Boc.

Fmoc-deprotection was achieved with 20% piperidine in NMP for 2×3 min. The coupling chemistry was DIC/HOAt/collidine in NMP. Amino acid/HOAt solutions (0.3 M/0.3 M in NMP at a molar excess of 3-10 fold) were added to the resin followed by the same molar equivalent of DIC (3 M in NMP) followed by collidine (3 M in NMP). For example, the following amounts of 0.3 M amino acid/HOAt solution were used per coupling for the following scale reactions: Scale/ml, 0.05 mmol/1.5 mL, 0.10 mmol/3.0 mL, 0.25 mmol/7.5 mL. Coupling time was either 2×30 min or 1×240 min.

Cleavage from the Resin

After synthesis the resin was washed with DCM, and the peptide was cleaved from the resin by a 2-3 hour treatment with TFA/TIS/water (95/2.5/2.5) followed by precipitation with diethylether. The precipitate was washed with diethylether.

### Purification and Quantification

The crude peptide is dissolved in a suitable mixture of water and MeCN such as water/MeCN (4:1) and purified by reversed-phase preparative HPLC (Waters Deltaprep 4000 or Gilson) on a column containing C18-silica gel. Elution is performed with an increasing gradient of MeCN in water containing 0.1% TFA. Relevant fractions are checked by analytical HPLC or UPLC. Fractions containing the pure target peptide are mixed and concentrated under reduced pressure. The resulting solution is analyzed (HPLC, LCMS) and the product is quantified using a chemiluminescent nitrogen specific HPLC detector (Antek 8060 HPLC-CLND) or by measuring UV-absorption at 280 nm. The product is dispensed into glass vials. The vials are capped with Millipore glassfibre prefilters. Freeze-drying affords the peptide trifluoroacetate as a white solid.

### Methods for Detection and Characterization

### LCMS Methods

### LCMS

LCMS was performed on a setup consisting of Waters Acquity UPLC system and LCT Premier XE mass spectrometer from Micromass. The UPLC pump was connected to two eluent reservoirs containing:

A: 0.1% Formic acid in water

B: 0.1% Formic acid in acetonitrile

The analysis was performed at RT by injecting an appropriate volume of the sample (preferably 2-10 μl) onto the column which was eluted with a gradient of A and B. The UPLC conditions, detector settings and mass spectrometer settings were:

Column: Waters Acquity UPLC BEH, C-18, 1.7 μm, 2.1 mm×50 mm

Gradient: Linear 5%-95% acetonitrile during 4.0 min (alternatively 8.0 min) at 0.4 ml/min

Detection: 214 nm (analogue output from TUV (Tunable UV detector))

MS ionisation mode: API-ES

Scan: 100-2000 amu (alternatively 500-2000 amu), step 0.1 amu

### UPLC Methods

### Method 05 B5 1

UPLC (method 05_B5_{—}1): The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C.

The UPLC system was connected to two eluent reservoirs containing:

A: 0.2 M Na₂SO₄, 0.04 M H₃PO₄, 10% CH₃CN (pH 3.5)

B: 70% CH₃CN, 30% H₂O

The following linear gradient was used: 60% A, 40% B to 30% A, 70% B over 8 minutes at a flow-rate of 0.35 ml/min.

### Method 04 A3 1

UPLC (method 04_A3_{—}1): The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C.

The UPLC system was connected to two eluent reservoirs containing:

A: 90% H₂O, 10% CH₃CN, 0.25 M ammonium bicarbonate

B: 70% CH₃CN, 30% H₂O

The following linear gradient was used: 75% A, 25% B to 45% A, 55% B over 16 minutes at a flow-rate of 0.35 ml/min.

### Method 04 A4 1

UPLC (method 04_A4_{—}1): The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40 ° C.

The UPLC system was connected to two eluent reservoirs containing:

A: 90% H₂O, 10% CH₃CN, 0.25 M ammonium bicarbonate

B: 70% CH₃CN, 30% H₂O

The following linear gradient was used: 65% A, 35% B to 25% A, 65% B over 16 minutes at a flow-rate of 0.35 ml/min.

### Method 08 B2 1

UPLC (method 08_B2_{—}1): The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C.

The UPLC system was connected to two eluent reservoirs containing:

A: 99.95% H₂O, 0.05% TFA

B: 99.95% CH₃CN, 0.05% TFA

The following linear gradient was used: 95% A, 5% B to 40% A, 60% B over 16 minutes at a flow-rate of 0.40 ml/min.

### Method 08 B4 1

UPLC (method 08_B4_{—}1): The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C.

The UPLC system was connected to two eluent reservoirs containing:

A: 99.95% H₂O, 0.05% TFA

B: 99.95% CH₃CN, 0.05% TFA

The following linear gradient was used: 95% A, 5% B to 5% A, 95% B over 16 minutes at a flow-rate of 0.40 ml/min.

### Method 10 B4 2

UPLC (method 08_B4_{—}1): The RP-analysis was performed using a Waters UPLC system fitted with a dual band detector. UV detections at 214nm and 254nm were collected using an ACQUITY UPLC BEH130, C18, 130 Å, 1.7 um, 2.1 mm×150 mm column, 50° C.

The UPLC system was connected to two eluent reservoirs containing:

A: 99.95% H₂O, 0.05% TFA

B: 99.95% CH₃CN, 0.05% TFA

The following linear gradient was used: 95% A, 5% B to 5% A, 95% B over 12 minutes at a flow-rate of 0.40 ml/min.

### Method 10 B5 2

UPLC (method 08_B4_{—}1): The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 50° C.

The UPLC system was connected to two eluent reservoirs containing:

A: 70% MeCN, 30% Water

B: 0.2M Na₂SO₄, 0.04 M H₃PO₄, 10% MeCN, pH 2.25

The following linear gradient was used: 40% A in 1 min, 40 -->70% A in 7 min at a flow-rate of 0.40 ml/min.

### Method 05 B7 1

UPLC (Method 05_B7_{—}1): The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C.

The UPLC system was connected to two eluent reservoirs containing:

A: 0.2 M Na₂SO₄, 0.04 M H₃PO₄, 10% CH₃CN (pH 3.5)

B: 70% CH₃CN, 30% H₂O

The following linear gradient was used: 80% A, 20% B to 40% A, 60% B over 8 minutes at a flow-rate of 0.40 ml/min.

### Method 04 A9 1

UPLC (Method 04_A9_{—}1): The RP-analysis was performed using a Waters UPLC system fitted with a dual band detector. UV detections at 214 nm and 254 nm were collected using an ACQUITY UPLC BEH Shield RP18, C18, 1.7 um, 2.1 mm×150 mm column, 60° C.

The UPLC system was connected to two eluent reservoirs containing:

A: 200 mM Na2SO4+20 mM Na2HPO4+20mM NaH2PO4 in 90% H₂O/10% CH3CN, pH 7.2

B: 70% CH₃CN, 30% H₂O

The following step gradient was used: 90% A, 10% B to 80% A, 20% B over 3 minutes, 80% A, 20% B to 50% A, 50% B over 17 minutes at a flow-rate of 0.40 ml/min.

### Example 1

[Glu²¹, Arg²⁵, Leu²⁷]Glucagon
H-H S Q G T F T S D Y S K Y L D S R R A Q E F V Q R L L N T-OH

The peptide was prepared essentially as described under the general methods.

UPLC 08_B2_{—}1: Rt=8.595 min; 98% purity.

UPLC 08_B4_{—}1: Rt=5.829 min; 100% purity.

LCMS: m/z=1150.1 (M+3H)3+, 862.9 (M+4H)4+, 690.5 (M+5)5+

### Example 2

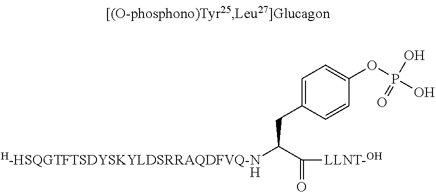

The peptide was prepared essentially as described under the general methods using Fmoc-Tyr(PO(NMe₂)₂)—OH (Novabiochem). The protected (O-phosphono)-tyrosine was deprotected by adding water to a total of 10% (VN) to the cleavage cocktail after cleavage from the resin.

The TFA-water mixture was kept for 16 hours to ensure deprotection of the phosphotyrosine.

UPLC 08_B2_{—}1: Rt=9.0

UPLC 08 B4_{—}1: Rt=6.1

UPLC 05_B5_{—}1: Rt=1.2

LCMS: m/z=1174.499 ((M/3)+3); 881.11 ((M/4)+4).

### Example 3

The glucagon analogue [Glu²¹,Arg²⁵,Leu²⁷]Glucagon (Example 1) was dissolved in 1 μM ThT, 10 mM glycylglycine buffer pH 4.0 to a concentration of 250 μM. This sample was subjected to the ThT fibrillation assay and incubated for 45 hours at 37° C. with vigorously shaking (960 rpm). Under these conditions no ThT emission occurred. Furthermore, the same concentration of glucagon analogue [Glu²¹,Arg²⁵,Leu²⁷]Glucagon (Example 1) was found in solution after completion of this assay as in the initial sample, i.e. recovery was 100%.

The analogues shown in table 2 were tested in the same assay. Results are depicted in the last two columns as lag time and recovery. No fibrillation was observed for any of the analogues.

### Example 4

The glucagon analogue [(O-phosphono)Tyr²⁵,Leu²⁷]Glucagon (Example 2) was dissolved to 250 μM in 1 μM ThT, 10 mM Hepes buffer pH 7.5. For comparison 290 μM glucagon, 50 mM glycin pH 2.5 and 1.45 mM glucagon, 50 mM glycin pH 2.5 (both containing 1 μM ThT) were included in the ThT fibrillation assay. The glucagon analogue [(O-phosphono)Tyr²⁵,Leu²⁷]Glucagon (example 2) had a lag time before fibrillation of approx. 16 hours. This is significantly longer than for native glucagon, having lag times of approx. 1.5 hours and less than half an hour at 290 μM and 1.45 mM, respectively.

### Example 5

[Arg²⁵, Leu²⁷]-Glucagon
H-H S Q G T F T S D Y S K Y L D S R R A Q D F V Q R L L N T-OH

The peptide was prepared essentially as described under the general methods.

UPLC 08_B2_{—}1: Rt=8.5 min; 98% purity

UPLC 04_A9_{—}1: Rt=5.1 min; 100% purity

LCMS: m/z=1145.2 (M+3H)3+, 859.1 (M+4H)4+, 687.5 (M+5)5+

### Example 6

[Glu²¹, Arg²⁵, Leu²⁷, Ser²⁸]-Glucagon
H-H S Q G T F T S D Y S K Y L D S R R A Q E F V Q R L L S T-OH

The peptide was prepared essentially as described under the general methods.

UPLC 08_B2_{—}1: Rt=8.8 min; 97% purity

UPLC 04_A9_{—}1: Rt=10,0 min; 100% purity

LCMS: m/z=1140.9 (M+3H)3+, 855.9 (M+4H)4+, 684.9 (M+5)5+

### Example 7

[His²⁵, Leu²⁷]-Glucagon
H-H S Q G T F T S D Y S K Y L D S R R A Q D F V Q H L L N T-OH

The peptide was prepared essentially as described under the general methods.

UPLC 08_B2_{—}1: Rt=8.2 min; 96% purity

UPLC 04_A9_{—}1: Rt=9.2 min; 96% purity

LCMS: m/z=1139.2 (M+3H)3+, 854.6 (M+4H)4+, 683.9 (M+5)5+

### Example 8

[Lys²⁵, Leu²⁷]-Glucagon
H-H S Q G T F T S D Y S K Y L D S R R A Q D F V Q K L L N T-OH

The peptide was prepared essentially as described under the general methods.

UPLC 05_B7_{—}1: Rt=5.3 min; 98% purity

UPLC 04_A9_{—}1: Rt=8.6 min; 100% purity

LCMS: m/z=1139.2 (M+3H)3+, 854.6 (M+4H)4+, 683.9 (M+5)5+

### Example 9

[Glu²¹, Lys²⁵, Leu²⁷]-Glucagon
H-H S Q G T F T S D Y S K Y L D S R R A Q E F V Q K L L N T-OH

The peptide was prepared essentially as described under the general methods.

UPLC 05_B7_{—}1: Rt=5.4 min; 98% purity

UPLC 04_A9_{—}1: Rt=9.3 min; 100% purity

LCMS: m/z=1140.6 (M+3H)3+, 855.7 (M+4H)4+, 684.7 (M+5)5+

### Example 10

[Glu¹⁵, Glu²¹, Arg²⁵, Leu²⁷]-Glucagon
H-H S Q G T F T S D Y S K Y L E S R R A Q E F V Q R L L N T-OH

The peptide was prepared essentially as described under the general methods.

UPLC 05_B7_{—}1: Rt=5.2 min; 100% purity

UPLC 04_A9_{—}1: Rt=7.9 min; 98% purity

LCMS: m/z=1154.5 (M+3H)3+, 866.1 (M+4H)4+, 693.1 (M+5)5+

### Example 11

[Arg²⁵, Leu²⁷, Glu²⁸]-Glucagon
H-H S Q G T F T S D Y S K Y L D S R R A Q D F V Q R L L E T-OH

The peptide was prepared essentially as described under the general methods.

UPLC 05_B7_{—}1: Rt=5.9 min; 100% purity

LCMS: m/z=1150.8 (M+3H)3+, 863.3 (M+4H)4+

### Pharmacological Methods

### Assay (I)

### Glucagon Activity

The glucagon receptor was cloned into HEK-293 cells having a membrane bound cAMP biosensor (ACTOne™). The cells (14000 per well) were incubated (37° C., 5% CO₂) overnight in 384-well plates. Next day the cells were loaded with a calcium responsive dye that only distributed into the cytoplasm. Probenecid, an inhibitor of the organic anion transporter, was added to prevent the dye from leaving the cell. A PDE inhibitor was added to prevent formatted cAMP from being degraded. The plates were placed into a FLIPR^{TETRA} and the glucagon analogues were added. End point data were collected after 6 minutes. An increase in intracellular cAMP was proportional to an increased in calcium concentrations in the cytoplasm. When calcium was bound the dry a fluorescence signal was generated. EC₅₀-values were calculated in Prism5.

TABLE 1
In vitro data on receptor binding

| | | Assay (I) |
|---|---|---|
| | | Glucagon |
| Example nr. | Molecule | [EC50] (nM) |
| Native | HumanGlucagon(1-29) | 0.003 |
| hGlucagon | | |
| Example 1 | [Glu²¹,Arg²⁵,Leu²⁷]Glucagon | 0.017 |
| Example 2 | [(O-phospho)Tyr²⁵,Leu²⁷]Glucagon | 0.017 |

TABLE 2
In vitro data on receptor binding and physical stability data

| | | Assay (I) | Assay (II) | Assay (II) |
|---|---|---|---|---|
| | | Glucagon | (pH 4.0) | (pH 4.0) |
| Example | | [EC50] | Lag time | Recovery |
| nr. | Peptide analogue | (nM) | (hours) | (%) |
| Native | Human glucagon (1-29) | 0.003 | 1.5 | 2.5 |
| hGlucagon | | | | |
| Example 5 | [Arg²⁵,Leu²⁷]-Glucagon | 0.057 | 45 | 96 |
| Example 6 | [Glu²¹,Arg²⁵,Leu²⁷,Ser²⁸]-Glucagon | 0.026 | 45 | 100 |
| Example 7 | [His²⁵,Leu²⁷]-Glucagon | 0.019 | 45 | 100 |
| Example 8 | [Lys²⁵,Leu²⁷]-Glucagon | 0.100 | 45 | 100 |
| Example 9 | [Glu²¹,Lys²⁵,Leu²⁷]-Glucagon | 0.043 | 45 | 100 |
| Example 10 | [Glu¹⁵,Glu²¹,Arg²⁵,Leu²⁷]-Glucagon | 0.266 | 45 | 100 |
| Example 11 | [Arg²⁵,Leu²⁷,Glu²⁸]-Glucagon | 0.037 | 45 | 100 |

### Assay (II)

### ThT Fibrillation Assays for the Assessment of Physical Stability of Protein Formulations

Low physical stability of a peptide may lead to amyloid fibril formation, which is observed as well-ordered, thread-like macromolecular structures in the sample eventually resulting in gel formation. This has traditionally been measured by visual inspection of the sample. However, that kind of measurement is very subjective and depending on the observer. Therefore, the application of a small molecule indicator probe is much more advantageous. Thioflavin T

(ThT) is such a probe and has a distinct fluorescence signature when binding to fibrils [Naiki et al. (1989) Anal. Biochem. 177, 244-249; LeVine (1999) Methods. Enzymol. 309, 274-284].
The time course for fibril formation can be described by a sigmoidal curve with the following expression [Nielsen et al. (2001) Biochemistry 40, 6036-6046]:

$$F = {f}_{i} + {m}_{i} t + \frac{{f}_{f} + {m}_{f} t}{1 + {e}^{- [ ( t - {t}_{0} ) / τ ]}} \qquad Eq . ( 1 )$$

Here, F is the ThT fluorescence at the time t. The constant t0 is the time needed to reach 50% of maximum fluorescence. The two important parameters describing fibril formation are the lag-time calculated by t0-2τ and the apparent rate constant kapp 1/τ.

Formation of a partially folded intermediate of the peptide is suggested as a general initiating mechanism for fibrillation. Few of those intermediates nucleate to form a template onto which further intermediates may assembly and the fibrillation proceeds. The lag-time corresponds to the interval in which the critical mass of nucleus is built up and the apparent rate constant is the rate with which the fibril itself is formed.

Samples were prepared freshly before each assay. Each sample composition is described in the legends. The pH of the sample was adjusted to the desired value using appropriate amounts of concentrated NaOH and HCl. Thioflavin T was added to the samples from a stock solution in H₂O to a final concentration of 1 μM.

Sample aliquots of 200 μl were placed in a 96 well microtiter plate (Packard OptiPlate™-96, white polystyrene). Usually, four or eight replica of each sample (corresponding to one test condition) were placed in one column of wells. The plate was sealed with Scotch Pad (Qiagen).

Incubation at given temperature, shaking and measurement of the ThT fluorescence emission were done in a Fluoroskan Ascent FL fluorescence platereader (Thermo Labsystems). The temperature was adjusted to the desired value, typically 30° C. or 37° C. The plate was either incubated without shaking (no external physical stress) or with orbital shaking adjusted to 960 rpm with an amplitude of 1 mm. Fluorescence measurement was done using excitation through a 444 nm filter and measurement of emission through a 485 nm filter.

Each run was initiated by incubating the plate at the assay temperature for 10 min. The plate was measured every 20 minutes for a desired period of time. Between each measurement, the plate was shaken and heated as described.

After completion of the ThT assay the four or eight replica of each sample was pooled and centrifuged at 20000 rpm for 30 minutes at 18° C. The supernatant was filtered through a 0.22 μm filter and an aliquot was transferred to a HPLC vial.

The concentration of peptide in the initial sample and in the filtered supernatant was determined by reverse phase HPLC using an appropriate standard as reference. The percentage fraction the concentration of the filtered sample constituted of the initial sample concentration was reported as the recovery.

The measurement points were saved in Microsoft Excel format for further processing and curve drawing and fitting was performed using GraphPad Prism. The background emission from ThT in the absence of fibrils was negligible. The data points are typically a mean of four or eight samples and shown with standard deviation error bars. Only data obtained in the same experiment (i.e. samples on the same plate) are presented in the same graph ensuring a relative measure of fibrillation between experiments.

The data set may be fitted to Eq. (1). However, the lag time before fibrillation may be assessed by visual inspection of the curve identifying the time point at which ThT fluorescence increases significantly above the background level.

1. A glucagon peptide comprising SEQ ID NO 1, wherein the amino acid residue at position 25 of said glucagon peptide, X₂₅, represents His, Lys, Ile, Leu, Ala, Met, Cys, Asn, Val, Ser, Gln, Asp, Glu, Thr or (p)Tyr;
or a pharmaceutically acceptable salt, amide, carboxylic acid or prodrug thereof.

2. The glucagon peptide according to claim 1, wherein the amino acid residue at position 25, i.e., X₂₅ represents His, Lys, or (p)Tyr;
or a pharmaceutically acceptable salt, amide, carboxylic acid or prodrug thereof.

3. A glucagon peptide comprising SEQ ID NO 1, wherein the amino acid residue at position 25 of said glucagon peptide, X₂₅, represents His, Arg, Lys, Ile, Leu, Ala, Met, Cys, Asn, Val, Ser, Gln, Asp, Glu, Thr or (p)Tyr;
or a pharmaceutically acceptable salt, amide, carboxylic acid or prodrug thereof, wherein said glucagon peptide is an agonist of the glucagon receptor.

4. The glucagon peptide according to claim 3, wherein the amino acid residue at position 25, i.e., X₂₅ represents His, Arg, Lys or (p)Tyr;
or a pharmaceutically acceptable salt, amide, carboxylic acid or prodrug thereof.

5. A glucagon peptide according to claim 1, wherein said glucagon peptide comprises up to ten amino acid residues substitutions as compared to human glucagon (1-29).

6. A glucagon peptide according to claim 5, wherein said up to ten substitutions are selected from the group of one or more amino acid positions consisting of X₃, X₂₀, X₂₄, X₁₆, X₁₇, X₁₈, X₂₁, X₂₇, X₂₈ and X₂₉.

7. A glucagon peptide according to claim 5, selected from the group consisting of:

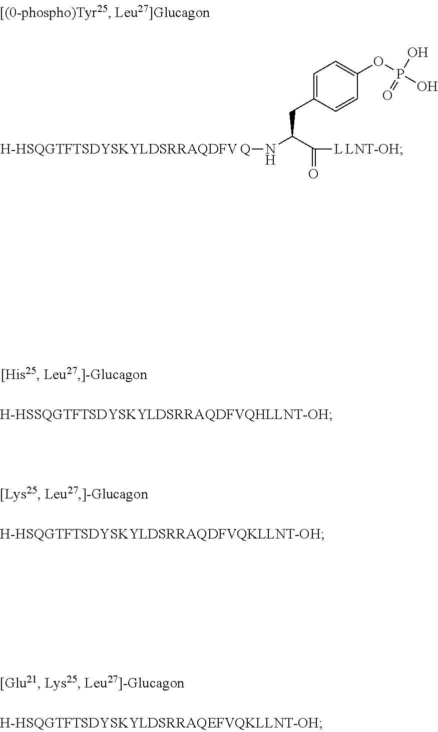

8. A pharmaceutical composition comprising a peptide according to claim 1.

9. The pharmaceutical composition according to claim 8, further comprising one or more additional therapeutically active compounds or substances.

10. The pharmaceutical composition according to claim 9, which is suited for parenteral administration.

11-15. (canceled)

16. A glucagon peptide according to claim 3, wherein said glucagon peptide comprises up to ten amino acid residues substitutions as compared to human glucagon (1-29).

17. A glucagon peptide according to claim 16, wherein said up to ten substitutions are selected from the group of one or more amino acid positions consisting of X₃, X₂₀, X₂₄, X_{16,} X₁₇, X₁₈, X₂₁, X₂₇, X₂₈ and X₂₉.

18. A glucagon peptide according to claim 16, selected from the group consisting of:

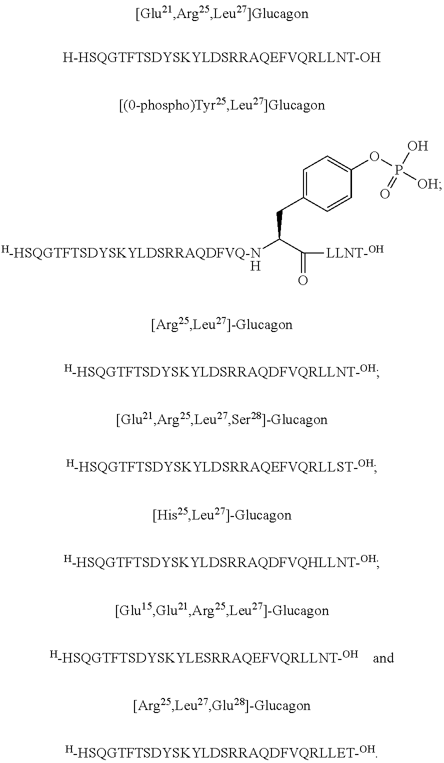

19. A pharmaceutical composition comprising a peptide according to claim 3.

20. A method of treating or preventing hypoglycemia, comprising administering to a patient in need thereof, an effective amount of a compound according to claim 1.

21. The method of claim 20, wherein in hypoglycemia is selected from the group consisting of insulin induced hypoglycemia, reactive hypoglycemia, diabetic hypoglycemia, non-diabetic hypoglycemia, fasting hypoglycemia, drug-induced hypoglycemia, gastric by-pass induced hypoglycemia, hypoglycemia in pregnancy, alcohol induced hypoglycemia, insulinoma and Von Girkes disease.

22. A method of treating or preventing hypoglycemia, comprising administering to a patient in need thereof, an effective amount of a compound according to claim 3.

23. The method of claim 22, wherein in hypoglycemia is selected from the group consisting of insulin induced hypoglycemia, reactive hypoglycemia, diabetic hypoglycemia, non-diabetic hypoglycemia, fasting hypoglycemia, drug-induced hypoglycemia, gastric by-pass induced hypoglycemia, hypoglycemia in pregnancy, alcohol induced hypoglycemia, insulinoma and Von Girkes disease.
